(19) United States
(12) Patent Application Publication
(10) Pub. No.: US 2026/0095085 A1
(43) Pub. Date: Apr. 2, 2026

(54) PLANAR DRIVE, PHARMACEUTICAL PLANT AND METHOD FOR DECONTAMINATING A PLANAR DRIVE

(71) Applicant: Syntegon Technology GmbH, Waiblingen (DE)

(72) Inventors: Thomas Kosian, Insingen (DE); Johannes Rauschnabel, Fichtenau (DE); Sebastian Schwarz, Neusitz (DE)

(21) Appl. No.: 19/103,900

(22) PCT Filed: Jul. 11, 2023
(86) PCT No.: PCT/EP2023/069101

§ 371 Date: Feb. 14, 2025

(30) Foreign Application Priority Data
Aug. 16, 2022 (DE) 10 2022 120 674.2

(51) Int. Cl. H02K 41/02; A61L 2/208; A61L 2/28; H02K 9/00

(52) U.S. Cl. CPC H02K 41/02; A61L 2/208; A61L 2/28; H02K 9/00

(57) ABSTRACT

23 The invention relates to a planar drive (10) with at least one stator (12) and at least one mover (14), wherein the planar drive (10) is designed to be resistant to hydrogen peroxide. The invention further relates to a pharmaceutical plant with said type of planar drive (10) and to a method for decontaminating said type of planar drive (10).

### BACKGROUND

The invention relates to a planar drive, to a pharmaceutical plant and to a method for decontaminating a planar drive.

A planar drive is a planar transport system with at least one mover, which can be moved in particular in a plane of movement while floating above a stator. The movement and the floating state of the mover occurs due to magnetic interaction between the mover and the stator. Such planar drives have not typically been used for aseptic applications because such applications place particularly high demands on hygiene. For example, aseptic applications usually require decontamination of the individual elements.

Due to their shape and the usually narrow gap between the mover and the stator, planar drives have the disadvantage that gaseous decontamination agents have little or no access. This can lead to the decontamination of these areas or surfaces not being possible or being possible only with a great deal of time and effort.

### SUMMARY

It is therefore an object of the present invention to provide a planar drive, a pharmaceutical plant and a method for decontaminating a planar drive, wherein use in aseptic applications is enabled.

The object is achieved by a planar drive according to the disclosure.

The planar drive comprises at least one stator having at least one first magnetic device for generating a first magnetic field. The stator can extend within a plane. In other words, the stator can have a flat, in particular planar, extension. The stator can in particular be designed to be planar.

The planar drive comprises at least one mover having at least one second magnetic device for generating a second magnetic field.

Due to magnetic interaction between the first magnetic field and the second magnetic field, the mover can be moved along a plane of movement oriented parallel to the stator (or to a side of the stator facing the mover). The distance or gap between the mover and the stator (or their surfaces facing one another) can be variable or adjustable.

The planar drive is designed to be resistant to hydrogen peroxide (H2O2). In other words, the planar drive is configured to be decontaminated using hydrogen peroxide. Due to its resistance to hydrogen peroxide, the planar drive can be decontaminated using hydrogen peroxide, enabling its use in aseptic applications.

For this purpose, for example, electrical components of the planar drive can be designed to be encapsulated. The planar drive can be made at least partially of stainless steel, in particular its outer surfaces can be made entirely of stainless steel. The individual (mechanically) connected elements of the planar drive can be firmly connected and/or sealed (e.g., by means of hydrogen-peroxide-resistant seals) such that no hydrogen peroxide can penetrate in particular at the contact or connection points of the respective (mechanically) connected elements.

A planar drive in the present context enables a movement of the mover in at least (in particular exactly) two dimensions within a plane of movement. Said plane of movement can be oriented parallel to the stator or to a side of the stator facing the mover.

The mover can be moved at a constant distance from the stator within the plane of movement. The distance of the mover can be influenced to a certain extent, which means that in such a case movements of the mover in a direction oriented perpendicular to the plane of movement are also possible. The extent of the movements perpendicular to the plane of movement (variation of the distance) is small compared to the movements in the plane of movement.

In the present case, a two-dimensional movement within the plane of movement means a free movement of the mover within the plane of movement. The mover can be moved to any position within the plane of movement. In the present case, free movement in the plane of movement means that the mover is not coupled or connected in particular to any rail or rail system (guided movement). In other words, the movement of the mover within the plane of movement is not restricted, in particular by means of a rail. This distinguishes the present concept of a planar drive in particular from a magnetic levitation train, which can be moved only along a rail, i.e., along a fixed movement path in a quasi one-dimensional manner (e.g., along a curved but still one-dimensionally extended rail). Movement transverse to the rail is not possible with such a train; only the distance to the rail can be varied slightly in some cases.

According to one development, the planar drive can comprise at least one cooling device associated with the mover. The cooling device can be arranged inside the mover. The cooling device can be designed to cool the mover. In particular, the cooling device can be configured for cooling below ambient temperature. What is provided for is, in particular, active cooling and not just passive cooling, e.g., by cooling fins. By cooling the mover, hydrogen peroxide from the environment of the mover can be deposited (or condensed or adsorbed) on the mover (or its surface).

Additionally or alternatively, the planar drive can comprise at least one cooling device associated with the stator. The cooling device can be arranged inside the stator. The cooling device can be designed to cool the stator. In particular, the cooling device can be configured for cooling below ambient temperature. What is provided for is, in particular, active cooling and not just passive cooling, e.g., by cooling fins. By cooling the stator, hydrogen peroxide from the environment of the stator can be deposited (or condensed or adsorbed) on the stator (or its surface).

By means of the cooling device for the mover or for the stator, a temperature (in particular on the surface of the mover and/or stator) can be set below the dew point, so that hydrogen peroxide can be deposited in a controlled manner on the mover and/or on the stator (or on their surfaces).

According to one development, the planar drive can comprise at least one heating device associated with the mover. The heating device can be arranged inside the mover. The heating device can be designed to heat (or warm) the mover. By heating the mover, the hydrogen peroxide deposited (or condensed or adsorbed) on the mover can be evaporated (or vaporized).

Additionally or alternatively, the planar drive can comprise at least one heating device associated with the stator. The heating device can be arranged inside the stator. The heating device can be designed to heat (or warm) the stator. By heating the stator, the hydrogen peroxide deposited (or condensed or adsorbed) on the stator can be evaporated (or vaporized).

Heating (or warming) the mover and/or the stator results, in particular, in a rapid evaporation or removal of the hydrogen peroxide from the surfaces of the mover and/or the stator.

According to one development, the planar drive can comprise at least one bioindicator. The bioindicator can be arranged on the mover and/or on the stator. A bioindicator can in particular be an element that indicates whether it has been exposed to hydrogen peroxide to an extent that is sufficient to kill biologically active substances, e.g., germs or spores.

A bioindicator can include a plate onto which spores have been dropped and dried. The spores are inactivated by hydrogen peroxide. This means that contact with hydrogen peroxide can be proven or verified, for example, by means of a spore growth test. This makes it possible to verify the exposure of certain surface sections to the hydrogen peroxide used for decontamination.

The plate can be designed to be round. The plate can be made of stainless steel. The bioindicator can be packed in or surrounded by a casing, in particular for handling the bioindicator. The casing can be designed to be permeable (pervious), in particular, to hydrogen peroxide. The casing can be made of a nonwoven fabric, in particular of high-density polyethylene (HDPE).

According to one development, the mover can have a recess on its side facing the stator for receiving or positioning the bioindicator. The bioindicator can thus be arranged at least partially, in particular completely) in the gap between the mover and the stator. This allows verification of whether decontamination has taken place within the gap or not.

According to one development, an indicator device removable from the mover can be arranged on the mover. The indicator device can comprise a bioindicator. The bioindicator can be located on or inside the indicator device. The indicator device can be attached to the mover for decontamination or to verify decontamination. After decontamination or verification of decontamination, the indicator device can be removed from the mover again.

The above object is further achieved by a pharmaceutical plant, in particular an aseptic isolator, according to the disclosure. The pharmaceutical plant, in particular the aseptic isolator, comprises a planar drive as described above.

As regards the advantages that can be achieved thereby, reference is made to the statements relating to the planar drive. The measures described in connection with the planar drive and/or the measures explained below can be used for the further design of the pharmaceutical plant.

The above object is further achieved by a method for decontaminating a planar drive according to the disclosure.

The planar drive comprises at least one stator having at least one first magnetic device for generating a first magnetic field.

The planar drive comprises at least one mover having at least one second magnetic device for generating a second magnetic field.

Due to magnetic interaction between the first magnetic field and the second magnetic field, the mover can be moved along a plane of movement oriented parallel to the stator (or to a side of the stator facing the mover). The distance or gap between the mover and the stator (or their surfaces facing one another) can be variable or adjustable.

The planar drive is arranged at least partially, in particular completely, within a work space that is closed off from its surroundings (or environment). The work space can be designed as an isolator.

The method comprises the steps of:

Providing the planar drive within the work space.

Introducing a decontamination agent, in particular a gaseous one, into the work space.

Moving the mover by means of magnetic interaction while the decontamination agent is within the work space.

By moving the mover, shaded areas or surfaces of a stationary mover, which the decontamination agent would have difficulty to reach or which it could not reach, can become accessible (or can be exposed). This allows the decontamination agent to reach the otherwise shaded areas of the planar drive.

This enables automatic decontamination of the planar drive within the work space (e.g., inside an isolator) without manual intervention by an operator. Accordingly, gloves ports for manual intervention in the work space can be dispensed with.

According to one development, the decontamination agent can be hydrogen peroxide (H2O2).

The method can further comprise the steps of:

Cooling the mover (or its surface) in order to deposit (or condense or adsorb) decontamination agent on the mover (or its surface).

Alternatively or additionally, cooling the stator (or its surface) in order to deposit (or condense or adsorb) decontamination agent on the stator (or its surface).

The method may comprise the steps of:

Heating the mover (or its surface) in order to remove (or evaporate or vaporize) decontamination agent from the mover (or its surface).

Alternatively or additionally, heating the stator (or its surface) in order to remove (or evaporate or vaporize) decontamination agent from the stator (or its surface).

According to one development, heating the mover (or its surface) can be implemented by increasing an electrical and/or magnetic current within the mover.

Alternatively or additionally, heating the stator (or its surface) can be implemented by increasing an electrical and/or magnetic current within the stator.

The method may comprise the step of:

Verifying decontamination by means of at least one bioindicator. The bioindicator can be arranged on the mover and/or the stator.

According to one development, a planar drive according to the above embodiments or a pharmaceutical plant according to the above embodiments can be used to carry out the method.

As regards the advantages that can be achieved thereby, reference is made to the statements relating to the planar drive or pharmaceutical plant. The measures described in connection with the planar drive or pharmaceutical plant and/or the measures explained below can be used for the further design of the method.

### BRIEF DESCRIPTION OF THE DRAWINGS

Further features, details and advantages of the invention emerge from the wording of the claims and from the following description of embodiments with reference to the drawings, in which, in each case schematically:

FIG. 1 shows a cross section through a planar drive according to a first exemplary embodiment;

FIG. 2 shows a view from below of a mover of the planar drive according to FIG. 1;

FIG. 3 shows a cross section through the planar drive according to a second exemplary embodiment;

FIG. 4 shows a view from below of the mover of the planar drive according to FIG. 3; and

FIG. 5 shows a flow chart illustrating a method for decontaminating the planar drive.

### DETAILED DESCRIPTION

In the following description and in the figures, corresponding components and elements bear the same reference signs. For improved clarity, not all reference signs are reproduced in all figures.

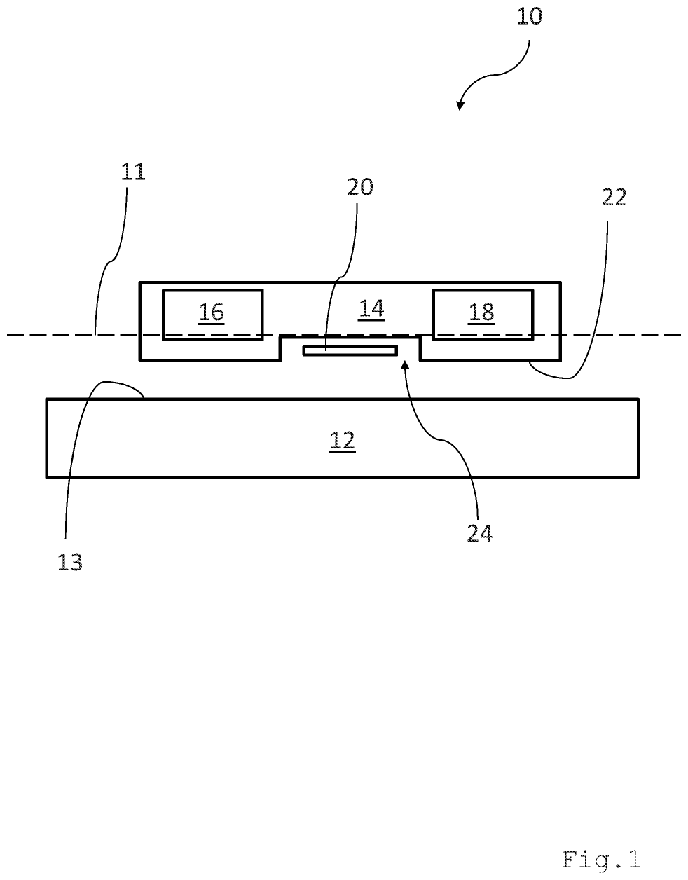

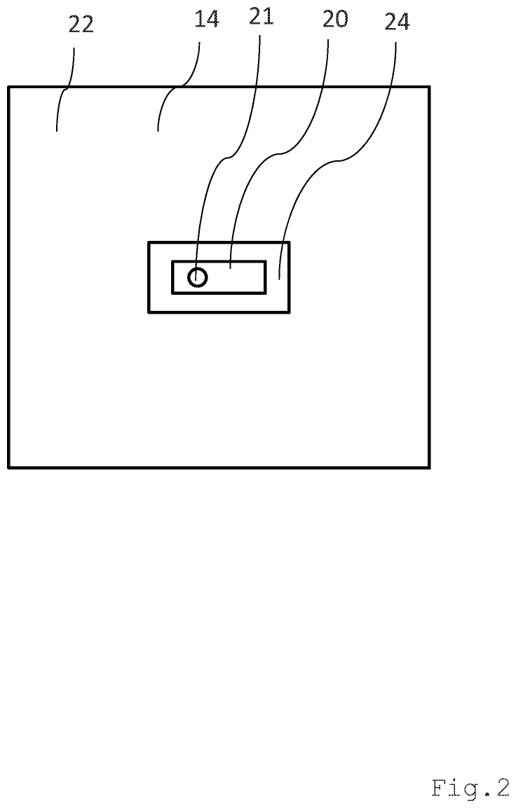

FIG. 1 shows a schematic cross section through a planar drive 10 according to a first exemplary embodiment. The planar drive 10 has a stator 12 and a mover 14. FIG. 2 shows a schematic view from below of the mover 14 of the planar drive 10 of FIG. 1. In the present case, the stator 12 and the mover 14 are each designed to be flat. In other words, the stator 12 and the mover 14 each have a planar extension.

The stator 12 has a first magnetic device (not shown) for generating a first magnetic field. The mover 14 has a second magnetic device (not shown) for generating a second magnetic field.

Due to magnetic interaction between the first magnetic field and the second magnetic field, the mover 14 can be moved freely within a plane of movement 11. In the present case, the plane of movement 11 is arranged parallel to a side 13 of the stator 12 facing the mover 14 and is indicated merely by dashed lines in FIG. 1.

The planar drive 10 is designed to be resistant to hydrogen peroxide. In other words, the planar drive 10 can be decontaminated using hydrogen peroxide as a decontamination agent.

The mover 14 has a cooling device 16. The cooling device 16 is arranged inside the mover 14 and configured to cool the mover 14. By cooling the mover 14, hydrogen peroxide from the environment of the mover 14 can be deposited on the mover 14 or its surface during decontamination.

It is conceivable for the stator 12 to also have a cooling device 16 in order to cool the stator 12 during decontamination and thus to deposit hydrogen peroxide from the environment of the stator 12 on the stator 12 or its surfaces.

The mover 14 further comprises a heating device 18. The heating device 18 is arranged inside the mover 14 and configured to heat or warm the mover. By heating the mover 14, the hydrogen peroxide deposited on the mover 14 or its surface can be evaporated or vaporized again after decontamination.

It is conceivable for the stator 12 to also have a heating device 18 in order to heat or warm the stator 12. By heating the stator 12, the hydrogen peroxide deposited on the stator 12 or its surface can be evaporated or vaporized again after decontamination.

In the present case, the mover 14 has a bioindicator 20. The bioindicator 20 has a round plate 21 onto which spores have been dropped and dried. The spores are inactivated upon contact with hydrogen peroxide. Complete inactivation is verified by growth control in a bacterial medium. Inactivated spores no longer show growth. The bioindicator 20 can thus be used to verify whether decontamination was successful, in particular at the location where the bioindicator 20 is located.

To accommodate the bioindicator 20, the mover 12 has a recess 24 on its side 22 facing the stator 12. The bioindicator 20 is arranged in the recess 24 of the mover 12. Since the gap between the mover 14 and the stator 12 or their sides 22, 13 facing one another represents a place that is difficult to reach or most difficult to reach by the hydrogen peroxide, arranging the bioindicator 20 in the recess 24 can serve to check whether decontamination was overall successful. If, for example, a bioindicator 20 arranged in the recess 24 does not show any growth in a bacterial medium, it can be assumed that other locations that can be reached more easily by the hydrogen peroxide have also been successfully decontaminated.

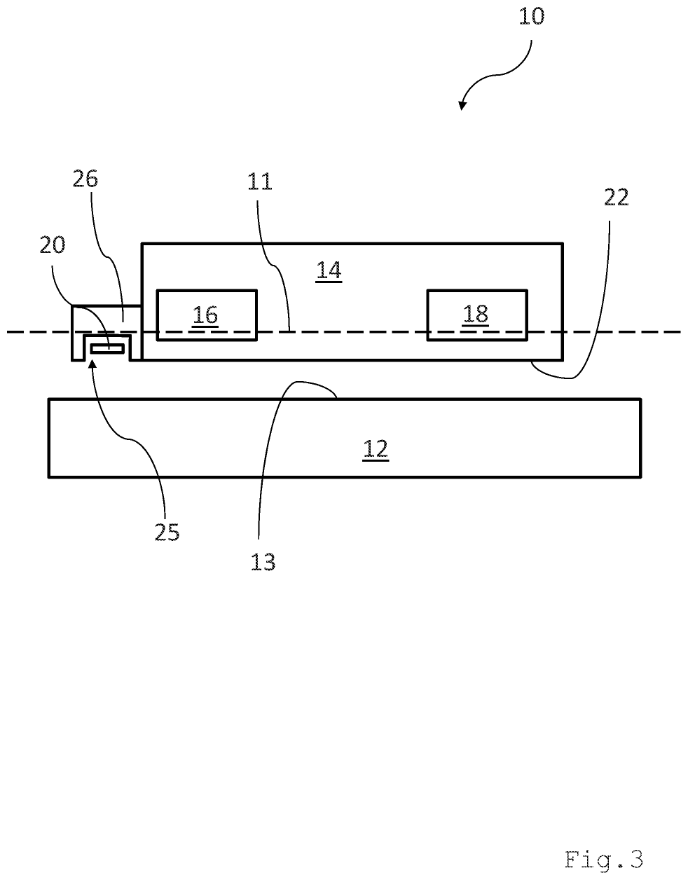

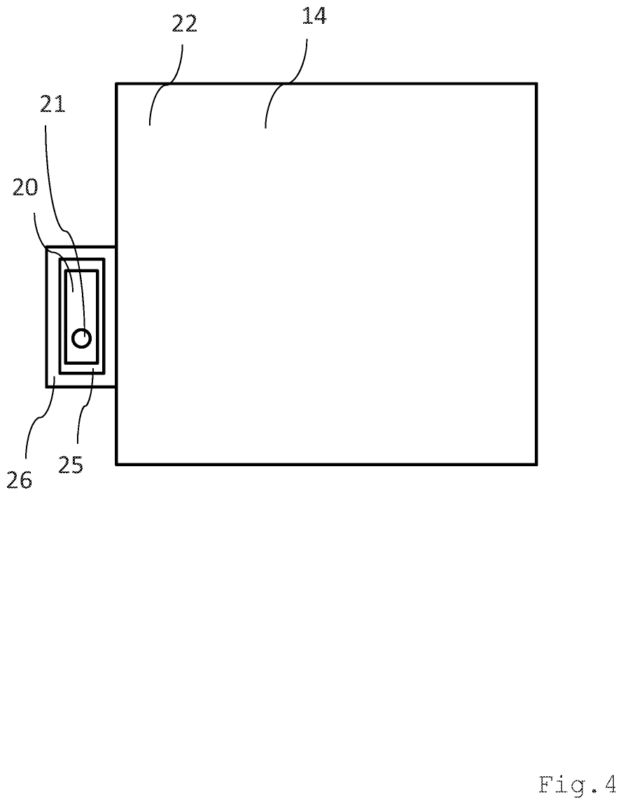

FIG. 3 shows a schematic cross section through the planar drive 10 according to a second exemplary embodiment and FIG. 4 shows a schematic view from below of the mover 14 of the planar drive 10 according to FIG. 3.

The second exemplary embodiment differs from the first exemplary embodiment in that the mover 14 does not have a recess 24 for accommodating the bioindicator 20. Instead, the mover 14 has an indicator device 26. In the present case, the indicator device 26 is arranged laterally, in a removable manner, on the mover 14. The indicator device 26 has an indicator receptacle 25 for accommodating the bioindicator 20.

The indicator device 26 can be attached to the mover 14 for decontamination (or for verification thereof) and removed from the mover 14 again after decontamination (or after verification thereof). The indicator device 26 can thus be removed from the mover 14 for proper operation of the planar drive 10.

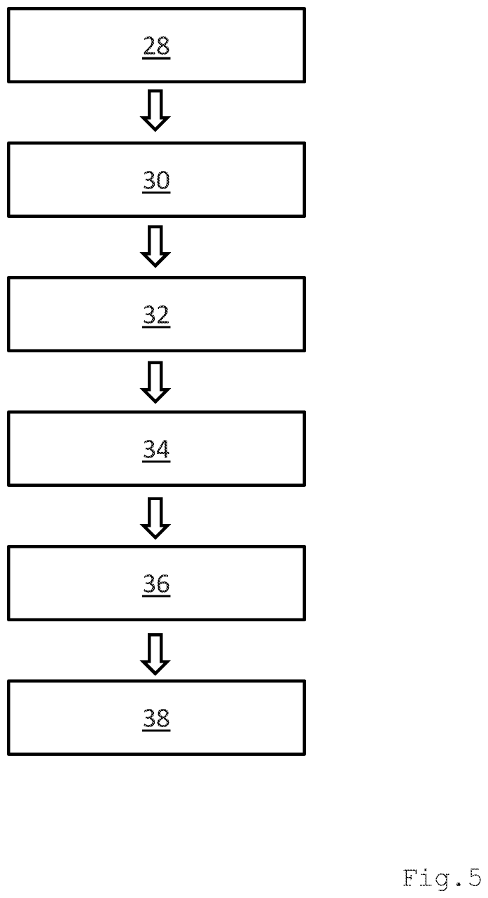

FIG. 5 shows a flow chart illustrating a method for decontaminating the planar drive 10.

For decontamination of the planar drive 10, the latter is arranged within a work space (not shown) that is closed off from its surroundings. This is indicated in FIG. 5 by means of a first method step 28.

To decontaminate the planar drive 10, a decontamination agent, in particular hydrogen peroxide, is introduced into the work space (second method step 30).

During and/or after the introduction of the decontamination agent into the work space, the mover 14 is moved by means of magnetic interaction (third method step 32).

By moving the mover 14 while the decontamination agent is located within the work space, the areas or surfaces that are switched off when the mover 14 is at a standstill are exposed. This allows the decontamination agent to also reach those places (e.g., within the gap between the stator 12 and the mover 14) of the planar drive 10 that would otherwise not be reached or would be difficult to reach (if the mover 14 is stationary).

In order to accelerate and/or improve the deposition or condensation of the decontamination agent on the mover 14 and/or stator 12, the mover 14 and/or the stator 12 are cooled (fourth method step 34).

The decontamination agent is removed after decontamination. To this end, the mover 14 and/or the stator 12 is warmed or heated (fifth method step 36). The higher temperature accelerates or promotes the evaporation or vaporization of the decontamination agent from the mover 14 and/or stator 12.

After removal of the decontamination agent, decontamination is verified using the bioindicator 20 (sixth process step 38). To this end, the bioindicator 20 can be placed at a location that is quite difficult for the decontamination agent to reach (e.g., within the gap between the mover 14 and the stator 12). If a bioindicator 20 positioned in this way shows a positive result, it can be assumed that decontamination was also successful at other locations that are more easily accessible for the decontamination agent.

1. A planar drive (10) comprising:
a stator (12) having at least one first magnetic device for generating a first magnetic field,
a mover (14) having at least one second magnetic device for generating a second magnetic field, wherein the mover (14) can be moved along a plane of movement (11) oriented parallel to the stator (12) due to magnetic interaction between the first magnetic field and the second magnetic field,
wherein the planar drive (10) is configured to be resistant to hydrogen peroxide.

2. The planar drive (10) according to claim 1, wherein the planar drive (10) comprises at least one cooling device (16) associated with the mover (14) in order to cool the mover (14), and/or wherein the planar drive (10) comprises at least one cooling device (16) associated with the stator (12) in order to cool the stator (12).

3. The planar drive (10) according to claim 1, wherein the planar drive (10) comprises at least one heating device (18) associated with the mover (14) in order to heat the mover (14), and/or wherein the planar drive (10) comprises at least one heating device (18) associated with the stator (12) in order to heat the stator (12).

4. The planar drive (10) according to claim 1, wherein the planar drive (10) comprises at least one bioindicator (20), wherein the bioindicator (20) is arranged on the mover (14) and/or on the stator (12).

5. The planar drive (10) according to claim 4, wherein the mover (14) has a recess (24) on a side (22) facing the stator (12) for receiving the bioindicator (20).

6. The planar drive (10) according to claim 1, wherein an indicator device (26) removable from the mover (14) is arranged on the mover (14), wherein the indicator device (26) comprises a bioindicator (20).

7. A pharmaceutical plant comprising a planar drive (10) according to claim 1.

8. A method for decontaminating a planar drive (10) comprising:
a stator (12) having at least one first magnetic device for generating a first magnetic field,
mover (14) having at least one second magnetic device for generating a second magnetic field, wherein the mover (14) can be moved along a plane of movement (11) oriented parallel to the stator (12) due to magnetic interaction between the first magnetic field and the second magnetic field, wherein the planar drive (10) is arranged at least partially within a work space that is closed off from its surroundings, the method comprising:
providing the planar drive (10) within the work space;
introducing a decontamination agent into the work space;
moving the mover (14) by magnetic interaction while the decontamination agent is within the work space.

9. The method according to claim 8, wherein the decontamination agent is hydrogen peroxide.

10. The method according to claim wherein the method further comprises:
cooling the mover (14) in order to deposit (34) decontamination agent on the mover (14) and/or
cooling the stator (12) in order to deposit (34) decontamination agent on the stator (12).

11. The method according to claim 8, wherein the method further comprises:
heating the mover (14) in order to remove (36) decontamination agent from the mover (14) and/or
heating the stator (12) in order to remove (36) decontamination agent from the stator (12).

12. The method according to claim 11, wherein heating the mover (14) is implemented by increasing an electrical and/or magnetic current within the mover (14), and/or wherein heating the stator (12) is implemented by increasing an electrical and/or magnetic current within the stator (12).

13. The method according to claim 8, wherein the method further comprises:
verifying decontamination by at least one bioindicator (20).

14. (canceled)

15. The planar drive (10) according to claim 1, wherein a distance between the mover (14) and the stator (12) is variable.

16. The planar drive (10) according to claim 2, wherein the at least one cooling device (16) associated with the mover (14) is arranged inside the mover (14), and/or wherein the at least one cooling device (16) associated with the stator (12) is arranged inside the stator (12).

17. The planar drive (10) according to claim 2, wherein the at least one cooling device (16) associated with the mover (14) is configured to deposit hydrogen peroxide from the environment of the mover (14) on the mover (14), and/or wherein the at least one cooling device (16) associated with the stator (12) is configured to deposit hydrogen peroxide from the environment of the stator (12) on the stator (12).

18. The planar drive (10) according to claim 3, wherein the at least one heating device (18) associated with the mover (14) is arranged inside the mover (14), and/or wherein the at least one heating device (18) associated with the stator (12) is arranged inside the stator (12).

19. The planar drive (10) according to claim 3, wherein the at least one heating device (18) associated with the mover (14) is configured to evaporate hydrogen peroxide deposited on the mover (14), and/or wherein the at least one heating device (18) associated with the stator (12) is configured to evaporate hydrogen peroxide deposited on the stator (12).

20. The method according to claim 8, wherein the decontamination agent is a gas.

21. The method according to claim 13, wherein the bioindicator (20) is arranged (38) on the mover (14) and/or the stator (12).
